(19) United States
(12) Patent Application Publication
(10) Pub. No.: US 2007/0249709 A1
(43) Pub. Date: Oct. 25, 2007

(54) Crystal forms of (S)-(8(2,6-dichlorophenyl)-6-fluoro-2,3-dihydro benzo[b][1,4]dioxin-2-yl)methanamine hydrochloride salt

(75) Inventors: Mahmoud Mirmehrabi, Laval (CA); Qing Yu, Laval (CA); Abdolsamad Tadayon, Kirkland (CA); Marc Tesconi, Monroe, NY (US); Yan Xu, New City, NY (US)

Correspondence Address: FISH & RICHARDSON P.C., P.O BOX 1022, MINNEAPOLIS, MN, 55440-1022, US

(73) Assignee: Wyeth, Madison, NJ (US)

(21) Appl. No.: 11/787,633

(22) Filed: Apr. 17, 2007

Related U.S. Application Data
(60) Provisional application No. 60/792,701, filed on Apr. 18, 2006.

(51) Int. Cl. C07D 319/14; A61K 31/335

(52) U.S. Cl. 514/452; 549/362

(57) ABSTRACT

The present invention is directed to crystal forms of the 5-HT_{2C} agonist (S)-(8-(2,6-dichlorophenyl)-6-fluoro-2,3-dihydrobenzo[b][1,4]dioxin-2-yl)methanamine hydrochloride, as well as compositions, processes of preparation, and uses thereof.

### CROSS REFERENCE TO RELATED APPLICATIONS

This application claims the benefit of U.S. Ser. No. 60/792,701, filed Apr. 18, 2006, the disclosure of which is incorporated herein in its entirety.

### FIELD OF THE INVENTION

The present invention is directed to crystal forms of the 5-HT_{2C} agonist (S)-(8-(2,6-dichlorophenyl)-6-fluoro-2,3-dihydrobenzo[b][1,4]dioxin-2-yl)methanamine hydrochloride salt, as well as compositions, processes of preparation, and uses thereof.

### BACKGROUND OF THE INVENTION

Schizophrenia affects approximately 5 million people. The most prevalent treatments for schizophrenia are currently the ‘atypical’ antipsychotics, which combine dopamine (D₂) and serotonin (5-HT_{2A}) receptor antagonism. Despite the reported improvements in efficacy and side-effect liability of atypical antipsychotics relative to typical antipsychotics, these compounds do not appear to adequately treat all the symptoms of schizophrenia and are accompanied by problematic side effects, such as weight gain (Allison, D. B., et. al., Am. J. Psychiatry, vol. 156, pp 1686-1696 (1999); Masand, P. S., Exp. Opin. Pharmacother. I: pp 377-389, (2000); Whitaker, R., Spectrum Life Sciences. Decision Resources. vol. 2, pp 1-9 (2000)).

Atypical antipsychotics also bind with high affinity to 5-HT_{2C} receptors and function as 5-HT_{2C} receptor antagonists or inverse agonists. Weight gain is a problematic side effect associated with atypical antipsychotics such as clozapine and olanzapine, and it has been suggested that 5-HT_{2C} antagonism is responsible for the increased weight gain. Conversely, stimulation of the 5-HT_{2C} receptor is known to result in decreased food intake and body weight (Walsh et. al., Psychopharmacology vol. 124, pp 57-73, (1996); Cowen, P. J., et. al., Human Psychopharmacology vol. 10, pp 385-391 (1995); Rosenzweig-Lipson, S., et. al., ASPET abstract (2000)).

Several lines of evidence support a role for 5-HT_{2C} receptor agonism or partial agonism as a treatment for schizophrenia. Studies suggest that 5-HT_{2C} antagonists increase synaptic levels of dopamine and may be effective in animal models of Parkinson's disease (Di Matteo, V., et. al., Neuropharmacology vol. 37, pp 265-272 (1998); Fox, S. H., et. al., Experimental Neurology vol. 151, pp 35-49 (1998)). Since the positive symptoms of schizophrenia are associated with increased levels of dopamine, compounds with actions opposite to those of 5-HT_{2C} antagonists, such as 5-HT_{2C} agonists and partial agonists, should reduce levels of synaptic dopamine. Recent studies have demonstrated that 5-HT_{2C} agonists decrease levels of dopamine in the prefrontal cortex and nucleus accumbens (Millan, M. J., et. al., Neuropharmacology vol. 37, pp 953-955 (1998); Di Matteo, V., et. al., Neuropharmacology vol. 38, pp 1195-1205 (1999); Di Giovanni, G., et. al., Synapse vol. 35, pp 53-61 (2000)), brain regions that are thought to mediate critical antipsychotic effects of drugs like clozapine. However, 5-HT_{2C} agonists do not decrease dopamine levels in the striatum, the brain region most closely associated with extrapyramidal side effects. In addition, a recent study demonstrates that 5-HT_{2C} agonists decrease firing in the ventral tegmental area (VTA), but not in the substantia nigra. The differential effects of 5-HT_{2C} agonists in the mesolimbic pathway relative to the nigrostriatal pathway suggest that 5-HT_{2C} agonists have limbic selectivity, and will be less likely to produce extrapyramidal side effects associated with typical antipsychotics.

Certain dihydrobenzodioxins are believed to be selective, potent agonists of the 5-HT_{2C} receptor and are therefore useful in a variety of applications, such as those recited above. The compound (S)-(8-(2,6-dichlorophenyl)-6-fluoro-2,3-dihydrobenzo[b][1,4]dioxin-2-yl)methanamine, shown below in Formula I, is an example of a dihydrobenzodioxin having such desirable characteristics. Preparation and characterization of this compound and its hydrochloric acid salt form are described in WO 2006/116158, which is incorporated herein by reference in its entirety. 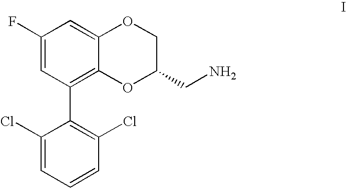
(S)-(8-(2,6-dichlorophenyl)-6-fluoro-2,3-dihydrobenzo[b][1,4]dioxin-2-yl)methanamine

Because improved drug formulations showing, for example, better bioavailability and/or better stability are consistently sought, there is an ongoing need for new or purer polymorphic forms of existing drug molecules. The crystal forms of (S)-(8-(2,6-dichlorophenyl)-6-fluoro-2,3-dihydrobenzo[b][1,4]dioxin-2-yl)methanamine hydrochloride described herein are directed toward this end.

### SUMMARY OF THE INVENTION

The present invention provides an anhydrous, non-solvated crystal form of (S)-(8-(2,6-dichlorophenyl)-6-fluoro-2,3-dihydrobenzo[b][1,4]dioxin-2-yl)methanamine hydrochloride designated as Form I.

The present invention further provides a hydrated form of (S)-(8-(2,6-dichlorophenyl)-6-fluoro-2,3-dihydrobenzo[b][1,4]dioxin-2-yl)methanamine hydrochloride.

The present invention further provides a hydrated crystal form of (S)-(8-(2,6-dichlorophenyl)-6-fluoro-2,3-dihydrobenzo[b][1,4]dioxin-2-yl)methanamine hydrochloride designated as Form II.

The present invention further provides a hydrated crystal form of (S)-(8-(2,6-dichlorophenyl)-6-fluoro-2,3-dihydrobenzo[b][1,4]dioxin-2-yl)methanamine hydrochloride designated as Form III.

The present invention further provides compositions comprising the hydrated forms and crystal forms described herein.

The present invention further provides methods of preparation of the hydrated forms and crystal forms described herein.

The present invention further provides hydrated forms and crystal forms prepared by the processes of preparation described herein.

The present invention further provides methods of treating 5-HT_{2C} associated diseases and conditions such as those recited herein.

### BRIEF DESCRIPTION OF THE DRAWINGS

FIG. 1 depicts an X-ray powder diffraction (XRPD) pattern characteristic of Form I.

FIG. 2 depicts a differential scanning calorimetry (DSC) thermogram characteristic of Form I.

FIG. 3 shows DVS cycle (mass change v. RH; starting from RH at 50%) characteristic of Form I.

FIG. 4 shows a DVS cycle (mass change v. RH; starting from RH at 0%) characteristic of Form I.

FIG. 5 depicts a thermal gravimetric analysis (TGA) thermogram characteristic of Form I.

FIG. 6 depicts an X-ray powder diffraction (XRPD) pattern characteristic of Form II.

FIG. 7 depicts a differential scanning calorimetry (DSC) thermogram characteristic of Form II.

FIG. 8 shows DVS cycle (mass change v. RH) characteristic of Form II.

FIG. 9 shows a DVS dynamic profile (mass change v. time) characteristic of Form II.

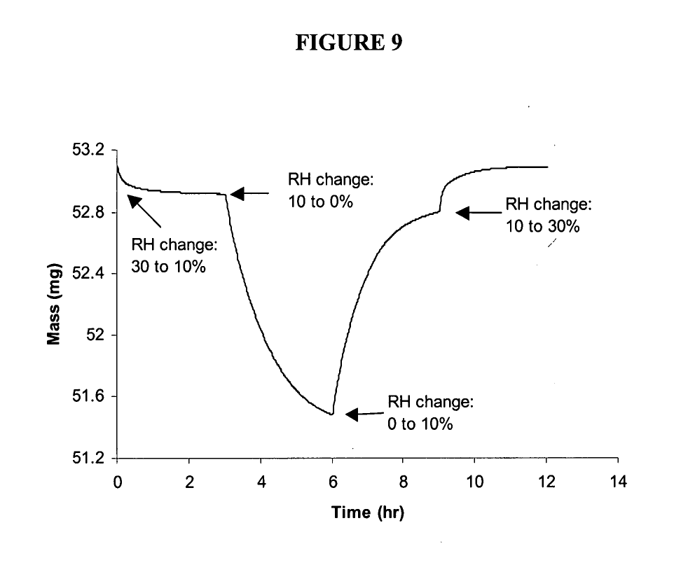

FIG. 10 depicts a thermal gravimetric analysis (TGA) thermogram characteristic of Form II.

FIG. 11 depicts an X-ray powder diffraction (XRPD) pattern characteristic of Form III.

FIG. 12 depicts a differential scanning calorimetry (DSC) thermogram characteristic of Form III.

FIG. 13 depicts a thermal gravimetric analysis (TGA) thermogram characteristic of Form III.

### DETAILED DESCRIPTION

Characterization of Crystal Forms

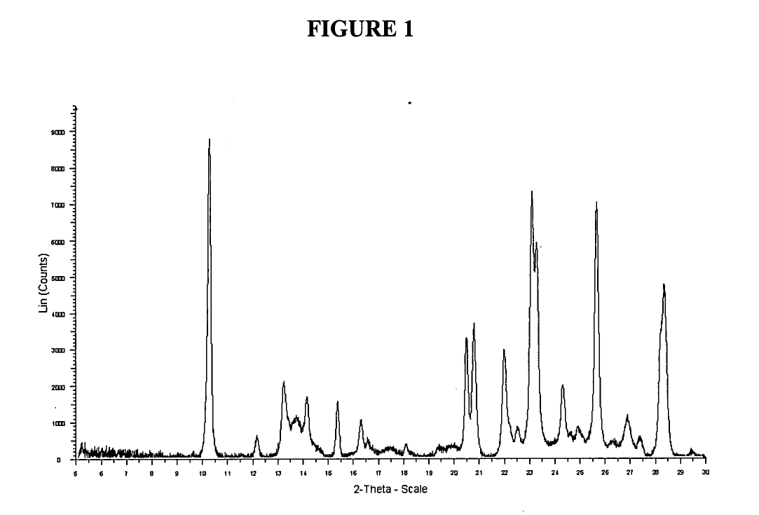

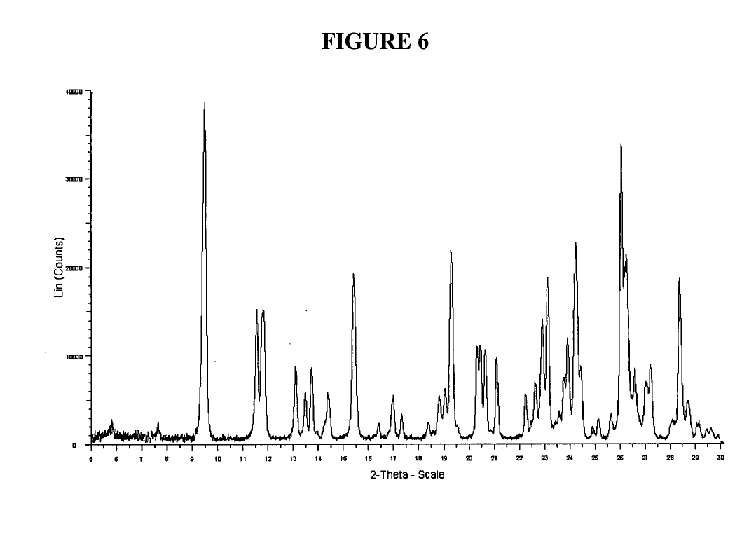

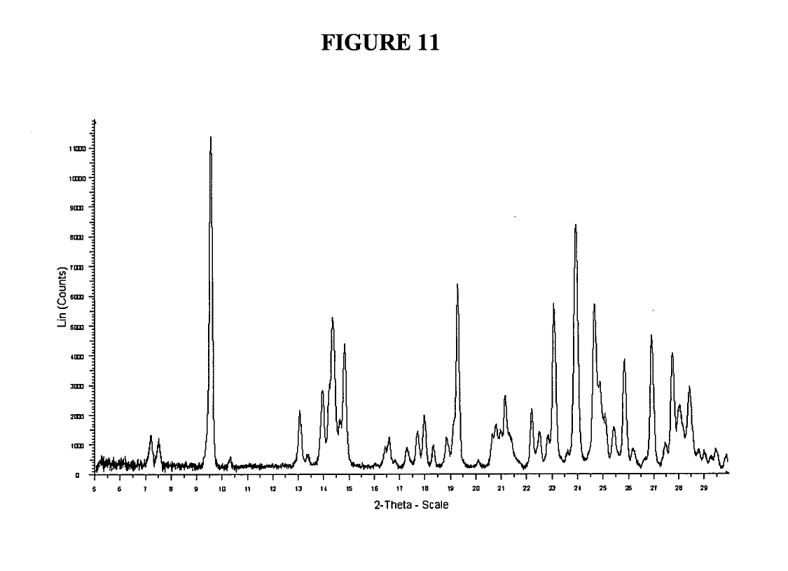

The present invention provides, inter alia, an anhydrous, non-solvated crystal form of the 5-HT_{2C} agonist (S)-(8-(2,6-dichlorophenyl)-6-fluoro-2,3-dihydrobenzo[b][1,4]dioxin-2-yl)-methanamine hydrochloride salt referred to herein as Form I. The present invention further provides crystal forms of hydrates of (S)-(8-(2,6-dichlorophenyl)-6-fluoro-2,3-dihydrobenzo[b][1,4]dioxin-2-yl)-methanamine hydrochloride which are referred to herein as Forms II and III. Each of the crystal forms can be identified by one or more solid state analytical methods such as X-ray powder diffraction (XRPD). For example, Form I can be identified by its powder X-ray diffraction pattern, an example of which is provided in FIG. 1; Form II can be identified by its powder X-ray diffraction pattern, an example of which is provided in FIG. 6; and Form III can be identified by its powder X-ray diffraction pattern, an example of which is provided in FIG. 11. Powder X-ray diffraction data consistent with Forms I, II and III are provided in Tables 1, 2 and 3 below respectively. Collection parameters for the X-ray data provided herein were as follows: voltage 40 kV; current 40.0 mA; 5.00-30.00 degree scan range; Bruker D8 Advance instrument; scan step size 0.01°; total scan time 30 minutes; using a Vantec-1 detector and Ni filter.

TABLE 1
(Form I)

| Degree (2θ) | Intensity (%) |
|---|---|
| 10.3 | 100.0 |
| 12.1 | 8.1 |
| 13.2 | 25.2 |
| 13.7 | 14.2 |
| 14.1 | 20.6 |
| 15.4 | 19.0 |
| 16.3 | 13.3 |
| 16.5 | 7.7 |
| 18.1 | 5.5 |
| 20.5 | 38.6 |
| 20.8 | 42.9 |
| 22.0 | 34.9 |
| 22.5 | 11.0 |
| 23.1 | 84.0 |
| 23.3 | 68.3 |
| 24.3 | 24.1 |
| 24.9 | 11.0 |
| 25.7 | 80.7 |
| 26.9 | 13.5 |
| 27.4 | 7.8 |
| 28.2 | 40.8 |
| 28.4 | 54.8 |

TABLE 2
(Form II)

| Degree (2θ) | Intensity (%) |
|---|---|
| 5.8 | 6.8 |
| 7.6 | 6.2 |
| 9.5 | 100.0 |
| 11.5 | 39.4 |
| 11.8 | 39.3 |
| 13.1 | 22.4 |
| 13.5 | 14.7 |
| 13.7 | 22.2 |
| 14.4 | 14.7 |
| 15.4 | 49.9 |
| 16.4 | 5.7 |
| 17.0 | 13.9 |
| 17.3 | 8.4 |
| 18.4 | 5.9 |
| 18.8 | 13.7 |
| 19.1 | 16.0 |
| 19.3 | 56.6 |
| 20.3 | 28.4 |
| 20.5 | 28.8 |
| 20.7 | 27.3 |
| 21.1 | 25.0 |
| 22.3 | 14.1 |
| 22.6 | 17.8 |
| 22.9 | 36.3 |
| 23.1 | 48.7 |
| 23.6 | 9.3 |
| 23.8 | 19.2 |
| 23.9 | 30.7 |
| 24.3 | 59.0 |
| 24.5 | 22.6 |
| 24.9 | 4.9 |
| 25.2 | 7.0 |
| 25.7 | 8.6 |
| 26.1 | 87.7 |
| 26.3 | 55.2 |
| 26.6 | 21.9 |
| 27.1 | 17.9 |
| 27.2 | 23.0 |
| 28.1 | 6.9 |
| 28.4 | 48.4 |
| 28.7 | 12.3 |
| 29.1 | 5.1 |
| 29.5 | 3.9 |
| 29.7 | 4.4 |

TABLE 3
(Form III)

| Degree (2θ) | Intensity (%) |
|---|---|
| 7.2 | 11.4 |
| 7.5 | 10.4 |
| 9.5 | 100.0 |
| 10.3 | 4.8 |
| 13.0 | 18.7 |
| 13.3 | 5.4 |
| 13.9 | 24.3 |
| 14.2 | 26.6 |
| 14.3 | 46.3 |
| 14.6 | 13.9 |
| 14.8 | 38.5 |
| 16.6 | 10.7 |
| 17.3 | 7.7 |
| 17.7 | 12.4 |
| 18.0 | 17.0 |
| 18.3 | 8.4 |
| 18.9 | 10.5 |
| 19.1 | 14.5 |
| 19.3 | 56.0 |
| 20.7 | 11.6 |
| 20.8 | 14.2 |
| 21.0 | 11.2 |
| 21.2 | 23.1 |
| 22.2 | 19.2 |
| 22.5 | 12.3 |
| 22.8 | 11.2 |
| 23.1 | 50.3 |
| 23.9 | 74.0 |
| 24.7 | 50.3 |
| 24.9 | 27.1 |
| 25.1 | 17.9 |
| 25.4 | 13.6 |
| 25.9 | 33.8 |
| 26.2 | 7.2 |
| 26.9 | 41.0 |
| 27.5 | 9.1 |
| 27.8 | 35.6 |
| 28.1 | 20.3 |
| 28.4 | 25.7 |
| 29.5 | 7.0 |
| 29.9 | 5.4 |

The relative intensities of the XRPD peaks can vary depending on, inter alia, the sample preparation technique, crystal size distribution, various filters used, the sample mounting procedure, and the particular instrument employed. Moreover, instrument variation and other factors can affect the 2-theta values. Therefore, the term “substantially” in the context of XRPD is meant to encompass that peak assignments can vary by plus or minus about 0.2°. Moreover, new peaks may be observed or existing peaks may disappear, depending on the type of the machine or the settings (for example, whether a Ni filter is used or not on a Bruker D8 Advance machine).

In some embodiments, Form I has a powder X-ray diffraction pattern comprising a characteristic peak, in terms of 2θ, at about 10.3° and at least one characteristic peak, in terms of 2θ, selected from about 23.1° and about 25.7°. In further embodiments, the powder X-ray diffraction pattern comprises characteristic peaks, in terms of 2θ, at about 10.3°, about 23.1°, and about 25.7°. In yet further embodiments, the powder X-ray diffraction pattern further comprises at least one characteristic peak, in terms of 2θ, selected from about 20.5° and about 20.8°. In yet further embodiments, the powder X-ray diffraction pattern further comprises a characteristic peak, in terms of 2θ, at about 22.0°. In some embodiments, the powder X-ray diffraction pattern comprises at least four characteristic peaks, in terms of 2θ, selected from about 10.3°, about 13.2°, about 14.1°, about 15.4°, about 20.5°, about 20.8°, about 22.0°, about 23.1°, about 23.3°, about 24.3°, about 25.7°, about 28.2° and about 28.4°. In further embodiments, Form I is characterized by a powder X-ray diffraction pattern substantially as shown in FIG. 1.

In some embodiments, Form II has a powder X-ray diffraction pattern comprising a characteristic peak, in terms of 2θ, at about 9.5° and at least one characteristic peak, in terms of 2θ, selected from about 15.4°, about 26.1°, and about 26.3°. In some further embodiments, the powder X-ray diffraction pattern comprises characteristic peaks, in terms of 2θ, at about 9.5°, about 15.4°, and about 26.1°. In yet further embodiments, the powder X-ray diffraction pattern comprises a characteristic peak, in terms of 2θ, selected from at about 11.5° and about 11.8°. In some embodiments, the powder X-ray diffraction pattern comprises at least four characteristic peaks, in terms of 2θ, selected from at about 9.5°, about 11.5°, about 11.8°, about 13.1°, about 13.7°, about 15.4°, about 19.3°, about 20.3°, about 20.5°, about 20.7°, about 21.10, about 22.90, about 23.1°, about 23.9°, about 24.3°, about 26.1°, about 26.3°, and about 28.4°. In further embodiments, Form II is characterized by a powder X-ray diffraction pattern substantially as shown in FIG. 6.

In some embodiments, Form III has a powder X-ray diffraction pattern comprising a characteristic peak, in terms of 2θ, at about 9.5° and at least one characteristic peak, in terms of 2θ, selected from about 14.3° and about 23.9°. In further embodiments, the diffraction pattern has characteristic peaks, in terms of 2θ, at about 9.5°, about 14.3°, and about 23.9°. In yet further embodiments, the diffraction pattern comprises a characteristic peak, in terms of 2θ, selected from at about 14.8°, about 24.7°, about 26.9°, and about 27.8°. In some embodiments, the powder X-ray diffraction pattern comprises at least four characteristic peaks, in terms of 2θ, selected from at about 9.5°, about 13.0°, about 13.9°, about 14.3°, about 14.8°, about 19.3°, about 20.3°, about 21.2°, about 22.9°, about 23.1°, about 23.9°, about 24.7°, about 25.9°, about 26.9°, about 27.3°, and about 28.4°. In some embodiments, Form III is characterized by a powder X-ray diffraction pattern substantially as shown in FIG. 11.

The crystal forms of the invention are readily distinguishable from each other by their spectroscopic and other characteristics. Sample data are compared for Forms I, II and III below in Table 4.

TABLE 4

| Measurement | Form I | Form II | Form III |
|---|---|---|---|
| DSC | Single Endotherm | Three Endotherm Peaks | Two Endotherm Peaks |
| | peak at: | at: | at: |
| | about 207° C. | about 94° C. (with onset at | about 112° C. (with |
| | (with onset at about | about 93° C. | onset at about 108° C.) |
| | 202° C.) | about 112° C. (with onset | about 208° C. (with |
| | | at about 104° C.) | onset at about 203° C.) |
| | | about 207° C. (with onset | |
| | | at about 201° C.) | |
| TGA (% weight | no substantial | about 4.2% loss in two | about 2.4% loss in one |
| loss) | weight loss | steps (about 2.4 wt %; then | step |
| | | about 1.8%) | |
| Example X-Ray | 10.3°, 15.4°, 27.0° | 9.5°, 11.5°, 11.8°, 15.4° | 9.5°, 14.8°, 24.7° |
| Powder peaks | | | |
| (2θ) | | | |
| Example | plates | Needles/rods | — |
| Crystal Habit | | | |

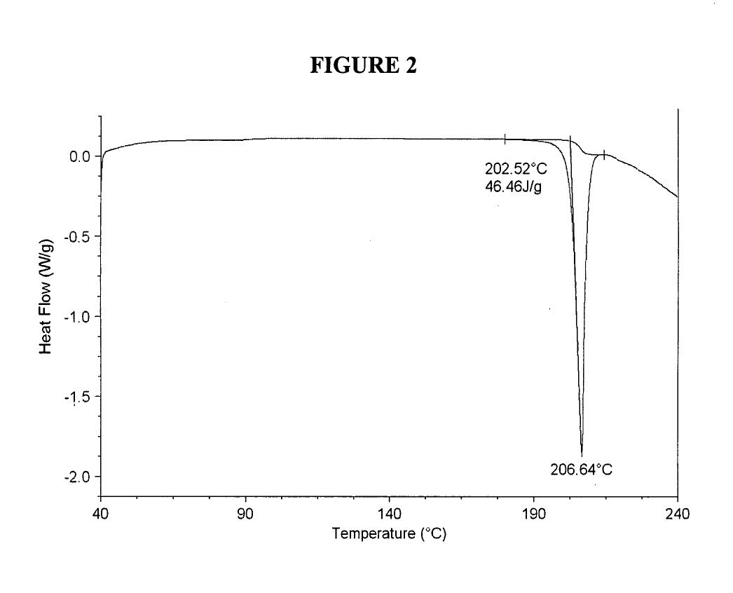

The DSC scans of Forms I, II and III are depicted in FIGS. 2, 7 and 12, respectively. Accordingly, the present invention provides a crystal form of the HCl salt of the compound of Formula I having a DSC thermogram substantially as shown in FIG. 2, 7, or 12. The location of DSC peaks obtained for Forms I, II and III may shift depending on, inter alia, the particle size distribution, the presence of impurities, the heating rate, and the type of the machine. Accordingly, the temperature reading can vary about ±4° C., and a crystal form having a DSC thermogram “substantially” as shown in the Figures is understood to accommodate such variation.

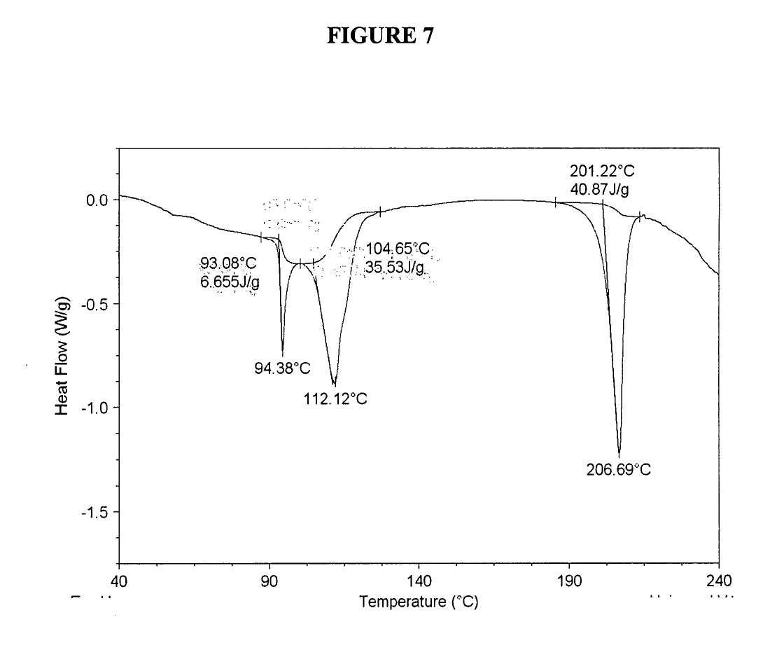

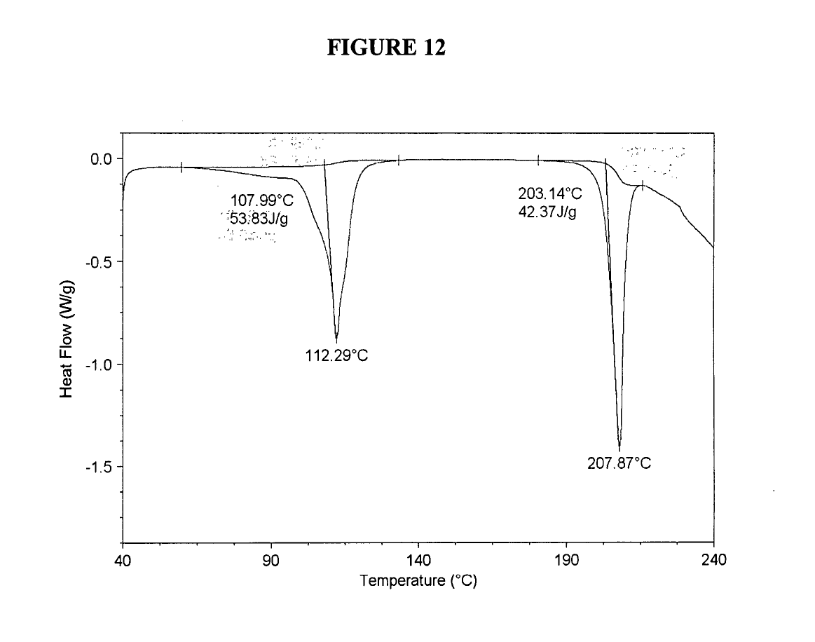

DSC data were collected using a TA instrument model Q1000 with the following parameters: 50 mL/min purge gas (N2); scan range 40 to 300° C., scan rate 10° C./min. As shown in FIG. 2, Form I is characterized by a strong endotherm at about 207° C. (onset temperature of about 202° C.) which is believed to be due to a melt event. As shown in FIG. 7, the DSC thermogram of Form II contains three endothermic peaks. The first peak occurs at about 94° C. (with onset temperature at about 93° C.), which is believed to correspond to a water loss event. The second peak occurs at about 112° C. (with an onset temperature at about 104° C.), which is also believed to correspond to a water loss event. The third peak occurs at about 207° C. (with an onset temperature at about 201° C.), which is believed to correspond to a melt event. As shown in FIG. 12, the DSC thermogram of Form III contains two endothermic peaks. The first peak occurs at about 112° C. (with an onset temperature at about 108° C.) which is believed to correspond to water loss. The second peak occurs at about 208° C. (with an onset temperature at about 203° C.), which is believed to correspond to a melt event.

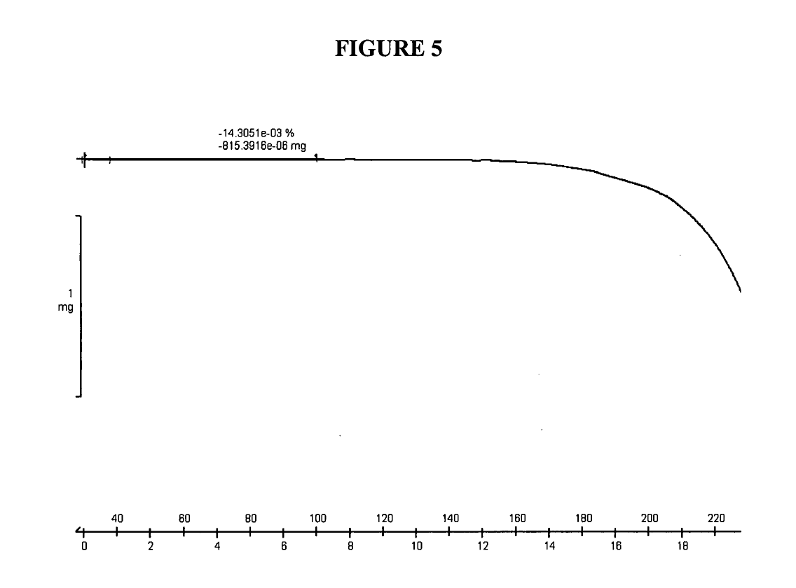

The TGA thermograms of Forms I, II and III are depicted in FIGS. 5, 10 and 13, respectively. Accordingly, the present invention provides a crystal form of the HCl salt of the compound of Formula I having a TGA thermogram substantially as shown in FIG. 5, 10, or 13. TGA data were collected using a TGA/SDTA 851^{e} (Mettler Toledo). A heating rate of 10° C./min between 30-300° C. was used and the TGA chamber was under 40 mL/min flow of nitrogen. The location and percentage of weight loss obtained for Forms I, II and III may shift depending on, inter alia, the particle size distribution, the presence of impurities, the heating rate, and the type of the machine. Accordingly, the weight loss reading can also vary slightly.

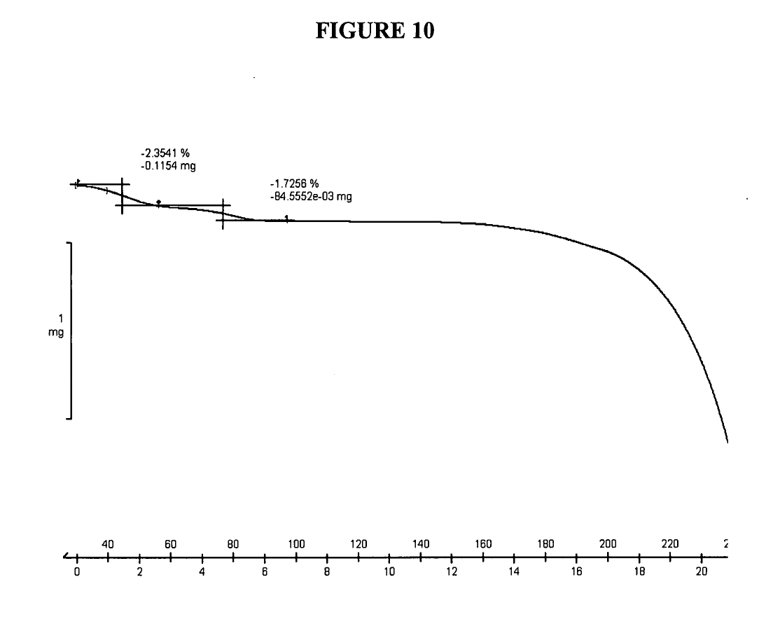

As shown in FIG. 5, the TGA thermogram of Form I shows little mass change. This result corroborates the assignment of Form I as a substantially anhydrous, non-solvated form. As shown in FIG. 10, the TGA thermogram of Form II shows about 4.2% weight loss in two steps (about 2.4% weight loss followed by about 1.8% weight loss). This is believed to indicate that Form II corresponds to a monohydrate (the theoretical hydrate-water content for a monohydrate of the salt is 4.7 wt %). However, the amount of hydrate water in Form II samples can vary depending on factors such as how the sample was prepared. In some embodiments, the molar ratio of the hydrate water to the (S)-(8-(2,6-dichlorophenyl)-6-fluoro-2,3-dihydrobenzo[b][1,4]dioxin-2-yl)methanamine hydrochloride salt is from about 0.5 to about 1.5, from about 0.5 to about 1.0, from about 0.8 to about 1.0, or about 1.0. In further embodiments, the water in Form II can be present in an amount of about 4.0 to about 6.5, about 4.5 to about 6.0, or about 4.5 to about 5.0 wt %.

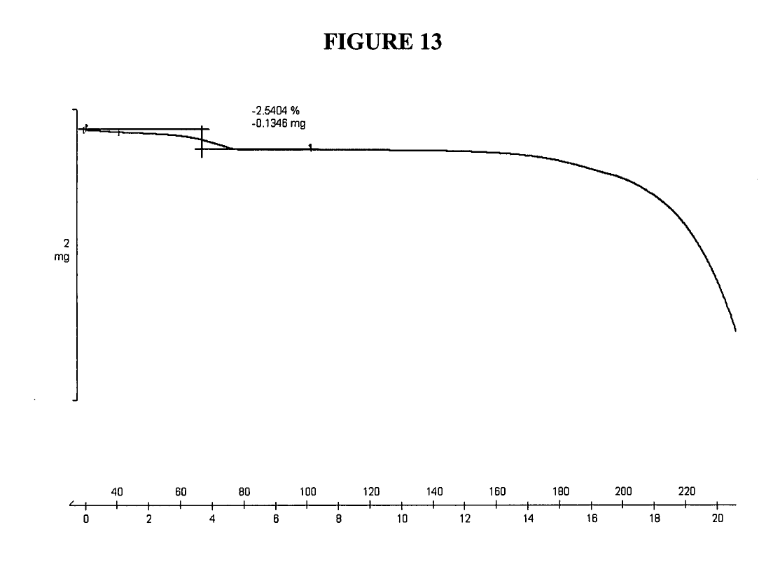

As shown in FIG. 13, the TGA thermogram of Form III shows about 2.4% weight loss in one step. This result indicates that Form III corresponds to a hemihydrate (the theoretical hydrate-water content for a hemihydrate of the salt is 2.4%). However, the amount of hydrate water in Form III samples can also vary depending on factors such as how the sample was prepared. In some embodiments, the molar ratio of the hydrate water to the (S)-(8-(2,6-dichlorophenyl)-6-fluoro-2,3-dihydrobenzo[b][1,4]dioxin-2-yl)methanamine hydrochloride salt is from about 0.3 to about 0.5, from about 0.4 to about 0.5, or about 0.5.

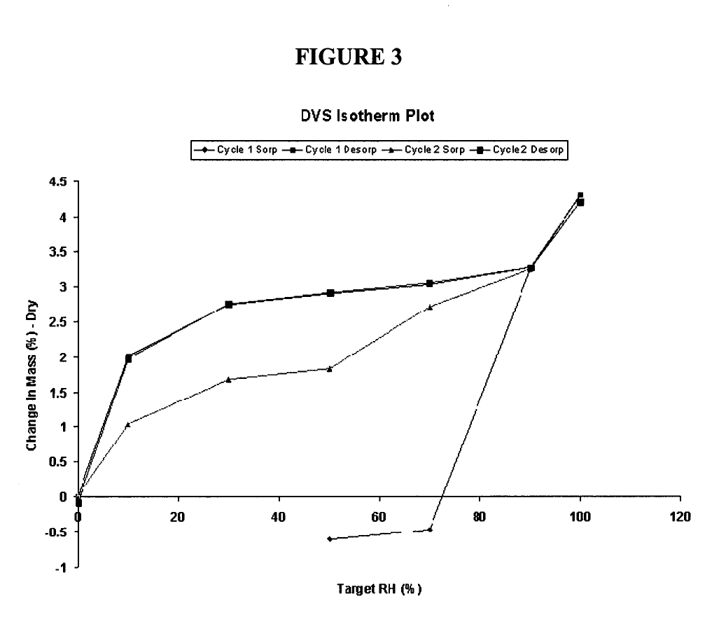

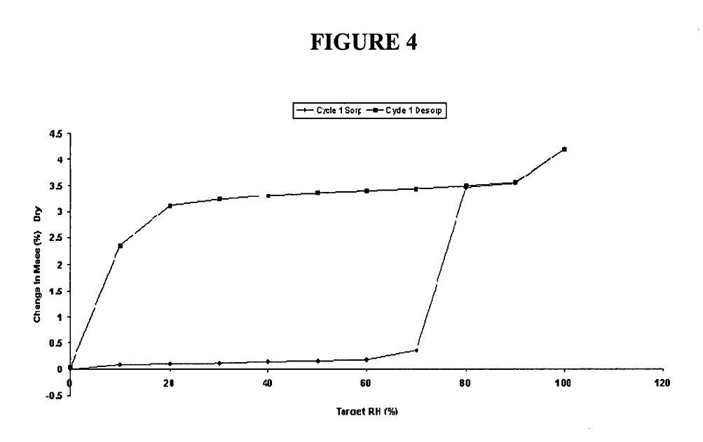

The hygroscopicity (the ability to absorb water vapor from the surrounding atmosphere) of Forms I and II was explored by dynamic vapor sorption (DVS) experiments. As shown in FIG. 3, a sample of Form I was subjected to various relative humidity (RH) at room temperature. The starting RH was about 50%, and little mass change was observed before reaching RH of 70%. When RH was scanned from 70% to 100%, significant mass change was observed (up to about 4.5%). When RH was scanned from 100% back to 50%, the mass change was found substantially irreversible (about 2.7%). When RH continued to be scanned from 50% to 0%, the sample lost water (weight). However, even when RH was at 0%, the sample weight did not return to its original amount. A second cycle of scanning indicated a new absorption behavior when RH was raised from 50% to 100%. This result indicates that Form I may be converted into another form at high RH. However, as shown in FIG. 4, a sample of Form I absorbed very little water when RH was raised from 0% only to about 70%.

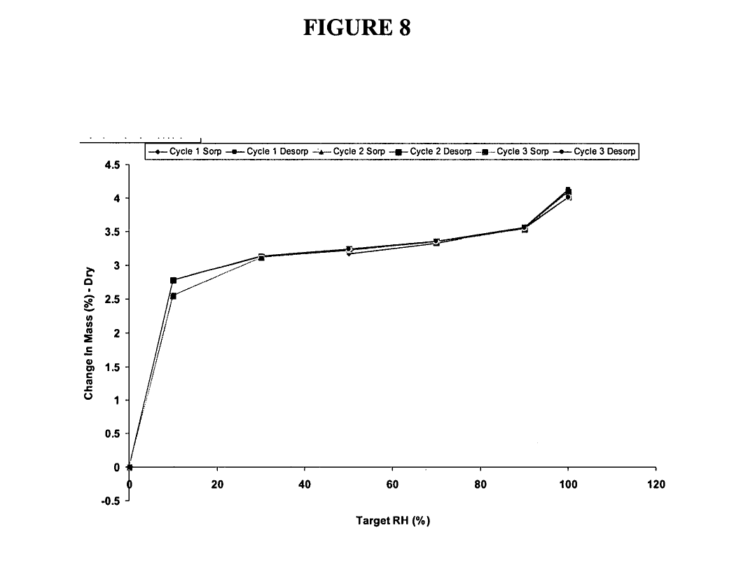

As shown in FIG. 8, a sample of Form II was subjected to various relative humidity (RH) at room temperature. The starting RH was about 50%, and only about 1% of mass change was observed when RH was scanned to 100% RH. When RH was scanned back to 50%, the mass change appeared reversible. The mass change was very slow when RH continued to be scanned from 50% to 10%. When RH was scanned from 10% to 0%, the sample lost about 2.7% of mass (water). A second scan cycle indicated that the observed mass change of Form II is largely reversible. This indicates that Form II is substantially stable even under high humidity (i.e., it does not appear to lose its crystal lattice integrity).

Additional experiments were carried out to assess the stabilities of Forms I, II and III with respect to exposure to solvents, heat, and physical force. Results are provided in Example 8.

The different properties discussed above contribute to numerous and readily apparent advantages of each of the forms. As an anhydrate, the final drug product of a preparation of Form I will likely be more consistent because it will not have variable water content observed in the hydrated forms. As hydrates, Forms II and III facilitate their preparation by eliminating the need for rigorously dry solvents and process conditions. Form II is stable under the widest of tested conditions and thus may have a superior shelf-life.

Methods of Preparation—Precipitation

The crystal forms of the invention can generally be prepared by any suitable method. In some embodiments, the forms can be precipitated from a solution of (S)-(8-(2,6-dichlorophenyl)-6-fluoro-2,3-dihydrobenzo[b][1,4]dioxin-2-yl)methanamine hydrochloride salt in a crystallizing solvent. The crystallizing solvent can contain any suitable organic solvent. In some embodiments, the crystallizing solvent is a polar organic solvent in which (S)-(8-(2,6-dichlorophenyl)-6-fluoro-2,3-dihydrobenzo[b][1,4]dioxin-2-yl)methanamine hydrochloride salt has appreciable solubility, such as greater than about 5 mg/mL at room temperature. In some embodiments, the solubility is greater than about 10 mg/mL, or is from about 10 mg/mL to about 500 mg/mL at room temperature. Example polar organic solvents include alcohols and esters.

Suitable alcohols include methanol, ethanol, 2-nitroethanol, 2-fluoroethanol, 2,2,2-trifluoroethanol, ethylene glycol, 1-propanol, isopropanol (2-propanol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, diethylene glycol monomethyl ether, diethylene glycol monoethyl ether, cyclohexanol, benzyl alcohol, phenol, glycerol, and the like. In some embodiments, the alcohol is isopropanol.

Suitable esters include alkyl esters such as ethyl acetate and isopropyl acetate.

In some embodiments, the crystallizing solvent can also contain various amounts of antisolvent such as a nonpolar or weakly-polar organic solvent in which (S)-(8-(2,6-dichlorophenyl)-6-fluoro-2,3-dihydrobenzo[b][1,4]dioxin-2-yl)methanamine hydrochloride salt has no significant solubility, such as less than about 2 mg/mL, at room temperature. Example non-polar or weakly polar organic solvents include ethers and hydrocarbons.

Suitable ethers include t-butylmethyl ether, diethyl ether, tetrahydrofuran, dimethoxymethane, 1,3-dioxane, 1,4-dioxane, furan, ethylene glycol dimethyl ether, ethylene glycol diethyl ether, diethylene glycol dimethyl ether, diethylene glycol diethyl ether, triethylene glycol dimethyl ether, anisole, and the like.

Suitable hydrocarbons include pentane, hexanes, heptanes, benzene, toluene, and the like.

In some embodiments, the crystallizing solvent contains t-butyl methyl ether (tBME).

In order to precipitate Form I from solution, the solution should be substantially free of water. For example, the solution can comprise less than about 1% water, less than about 0.5%, or less than about 0.2% water by weight.

In order to precipitate Forms II and III from the solution, the solution should contain water. In some embodiments, Form II is precipitated from the solution containing water. In some embodiments, the molar ratio of the water in the solution to (S)-(8-(2,6-dichlorophenyl)-6-fluoro-2,3-dihydrobenzo[b][1,4]dioxin-2-yl)methanamine hydrochloride is greater than about 3. In some embodiments, the solution contains at least about 2.5% by volume of water, at least about 5% by volume of water, at least about 10% by volume of water, at least about 20% by volume of water, at least about 50% by volume of water, at least about 75% by volume water, or at least about 99% water. In some embodiments, the solution contains at least about 2.5% by volume of water.

In some embodiments, Form III can be prepared by precipitation from a solution comprising (S)-(8-(2,6-dichlorophenyl)-6-fluoro-2,3-dihydrobenzo[b][1,4]dioxin-2-yl)methanamine hydrochloride and water. In some embodiments, the precipitation is carried out by reducing the amount of the water at a temperature of about 40° C. to about 60° C., about 45° C. to about 55° C., or about 50° C. In some embodiments, the reducing is carried out by evaporation under reduced pressure.

Precipitation of the crystal forms of the invention can be carried out by any suitable manner. For example, solutions of (S)-(8-(2,6-dichlorophenyl)-6-fluoro-2,3-dihydrobenzo[b][1,4]dioxin-2-yl)methanamine hydrochloride salt can be evaporated, cooled, treated with antisolvent, or combinations thereof. Treatment with antisolvent can be carried out by layering or vapor diffusion techniques. Suitable antisolvents include organic solvents that are miscible with the crystallizing solvent, yet are relatively poor solvents for the subject compound.

In some embodiments, the crystal forms of the invention are precipitated by reducing the volume of solution (i.e., increasing concentration of the compound of Formula I). In some embodiments, the reducing of volume is carried out by evaporation such as, for example, under reduced pressure.

In some embodiments, the precipitation is induced by cooling the solution. In further embodiments, the cooling is carried out at a rate of less than about 2θ° C./hour, less than about 15° C./hour, or less than about 10° C./hour.

Methods of Preparation—Reactive Crystallization

Forms I and II can be prepared by combining hydrogen chloride with a first solution of a compound of (S)-(8-(2,6-dichlorophenyl)-6-fluoro-2,3-dihydrobenzo[b][1,4]dioxin-2-yl)methanamine in a suitable first organic solvent. In some further embodiments, the first organic solvent is a non-polar or weakly polar solvent such as an ether. In yet further embodiments, the solvent contains tert-butyl methyl ether.

In order to generate Form I, the combining is carried out under substantially anhydrous conditions. For example, the solvents used to dissolve the free base and to introduce the hydrogen chloride contain about 1% or less, about 0.5% or less, or about 0.25% or less water by weight.

In some embodiments for the preparation of Form I, the hydrogen chloride is optionally present in a second organic solvent prior to the combining. In some further embodiments, the second organic solvent is an alcohol such as isopropanol.

In order to generate Form II, the combining is carried out in the presence of water. For example, the solution containing the dissolved free base together with the hydrogen chloride contains at least about 2.5%, at least about 5%, at least about 10%, at least about 20%, at least about 50%, at least about 75%, or at least about 99% by weight of water.

In some embodiments for the preparation of Form II, the hydrogen chloride is provided as an aqueous solution in the combining.

The hydrogen chloride in the preparation of either Forms I or II can be provided in any suitable amount including molar excess relative to the free amine. In some embodiments, the molar ratio of the hydrogen chloride to the compound of (S)-(8-(2,6-dichlorophenyl)-6-fluoro-2,3-dihydrobenzo[b][1,4]dioxin-2-yl)methanamine is from about 1 to about 2, about 1 to about 1.5, or about 1 to about 1.2.

Methods of Preparation—Other

Crystal Form I of the invention can also be prepared by slurrying (S)-(8-(2,6-dichlorophenyl)-6-fluoro-2,3-dihydrobenzo[b][1,4]dioxin-2-yl)methanamine hydrochloride or a hydrate thereof in an organic solvent, wherein the organic solvent contains little or is substantially free of water. In some embodiments, the organic solvent contains about 1% or less, about 0.5% or less, or about 0.2% or less by weight of water. In some embodiments, the organic solvent comprises ethanol, isopropanol, ethyl acetate, tert-butyl methyl ether, or acetonitrile.

Form I of the invention can further be prepared by heating (S)-(8-(2,6-dichlorophenyl)-6-fluoro-2,3-dihydrobenzo[b][1,4]dioxin-2-yl)methanamine hydrochloride (including hydrates or mixtures thereof) at elevated temperature (e.g., greater than about 50° C.). In some embodiments, the elevated temperature is from about 50° C. to about 150° C., about 50° C. to about 120° C., or about 50° C. to about 110° C. The heating can be carried out at any suitable pressure such as ambient pressure or a reduced pressure for a time sufficient to afford Form I.

Crystal Form II of the invention can also be prepared by slurrying (S)-(8-(2,6-dichlorophenyl)-6-fluoro-2,3-dihydrobenzo[b][1,4]dioxin-2-yl)methanamine hydrochloride (hydrates and mixtures thereof) in solvent containing water. The solvent can contain at least about 2.5%, at least about 5%, at least about 10%, at least about 20%, at least about 50%, at least about 75%, or at least about 99% by weight of water. In some embodiments, the solvent is water. In some embodiments, the slurrying is carried out at room temperature.

Compositions

The present invention further provides compositions containing a crystal form of the invention. In some embodiments, at least about 50%, at least about 70%, at least about 80%, at least about 90%, at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of total (S)-(8-(2,6-dichlorophenyl)-6-fluoro-2,3-dihydrobenzo[b][1,4]dioxin-2-yl)methanamine hydrochloride (or hydrate thereof) in a composition is present as Form I, Form II or Form III. In further embodiments, compositions of the present invention consist essentially of (S)-(8-(2,6-dichlorophenyl)-6-fluoro-2,3-dihydrobenzo[b][1,4]dioxin-2-yl)methanamine hydrochloride salt (or hydrate thereof) where at least about 95%, at least about 97%, at least about 98.0%, at least about 98.1%, at least about 98.2%, at least about 98.3%, at least about 98.4%, at least about 98.5%, at least about 98.6%, at least about 98.7%, at least about 98.8%, at least about 98.9%, at least about 99.0%, at least about 99.1%, at least about 99.2%, at least about 99.3%, at least about 99.4%, at least about 99.5%, at least about 99.6%, at least about 99.7%, at least about 99.8%, or at least about 99.9% by weight of the (S)-(8-(2,6-dichlorophenyl)-6-fluoro-2,3-dihydrobenzo[b][1,4]dioxin-2-yl)methanamine hydrochloride (or hydrate thereof) is present in the composition as either Form I, Form II or Form III. In some embodiments, the remainder (S)-(8-(2,6-dichlorophenyl)-6-fluoro-2,3-dihydrobenzo[b][1,4]dioxin-2-yl)methanamine hydrochloride salt (including both anhydrous salt and hydrate) is present as amorphous material or other crystal form. In some embodiments, the composition contains a mixture of Forms I, Form II and III, any subset thereof. Respective amounts of crystal forms of (S)-(8-(2,6-dichlorophenyl)-6-fluoro-2,3-dihydrobenzo[b][1,4]dioxin-2-yl)methanamine hydrochloride (including both anhydrous salt and hydrate) in a composition can be determined by any suitable spectroscopic method, such as X-ray powder diffraction.

Methods of Use

The crystal forms of the invention are useful as 5-HT_{2C} agonists in methods of treating, for example, schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease.

The crystal forms of the invention are further useful in methods for treating bipolar disorders, depressive disorders, mood episodes, anxiety disorders, adjustment disorders, or eating disorders. In some embodiments, the bipolar disorder is bipolar I disorder, bipolar II disorder, or cyclothymic disorder; the depressive disorder is major depressive disorder, dysthymic disorder, or substance-induced mood disorder; the mood episode is major depressive episode, manic episode, mixed episode, or hypomanic episode; the anxiety disorder is panic attack, agoraphobia, panic disorder, specific phobia, social phobia, obsessive compulsive disorder, posttraumatic stress disorder, acute stress disorder, generalized anxiety disorder, separation anxiety disorder, or substance-induced anxiety disorder.

The crystal forms of the invention are further useful in methods for treating pain, urinary incontinence, substance abuse, addiction to alcohol and other drugs including cocaine and nicotine, epilepsy, sleep disorders, migraines, sexual dysfunction, gastrointestinal disorders, or obesity.

The crystal forms of the invention are further useful in methods for treating central nervous system deficiency associated with trauma, stroke, or spinal cord injury.

Methods of treating the diseases listed herein are understood to involve administering to a patient in need of such treatment a therapeutically effective amount of the crystal form of the invention, or composition containing the same. As used herein, the term “treating” in reference to a disease is meant to refer to preventing, inhibiting and/or ameliorating the disease.

As used herein, the term “patient” refers to any animal, including mammals, preferably mice, rats, other rodents, rabbits, dogs, cats, swine, cattle, sheep, horses, or primates, and most preferably humans.

As used herein, the phrase “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following:

(1) preventing the disease; for example, preventing a disease, condition or disorder in an individual that may be predisposed to the disease, condition or disorder but does not yet experience or display the pathology or symptomatology of the disease;

(2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and

(3) ameliorating the disease; for example, ameliorating a disease, condition or disorder in an individual that is experiencing or displaying the pathology or symptomatology of the disease, condition or disorder (i.e., reversing the pathology and/or symptomatology).

In certain embodiments, the invention relates to compositions comprising at least one crystal form of the invention, and one or more pharmaceutically acceptable carriers, excipients, or diluents. Such compositions are prepared in accordance with acceptable pharmaceutical procedures, such as, for example, those described in Remingtons Pharmaceutical Sciences, 17th edition, ed. Alfonoso R. Gennaro, Mack Publishing Company, Easton, Pa. (1985), which is incorporated herein by reference in its entirety. Pharmaceutically acceptable carriers are those carriers that are compatible with the other ingredients in the formulation and are biologically acceptable.

The crystal forms of the invention can be administered orally or parenterally, neat, or in combination with conventional pharmaceutical carriers. Applicable solid carriers can include one or more substances that can also act as flavoring agents, lubricants, solubilizers, suspending agents, fillers, glidants, compression aids, binders, tablet-disintegrating agents, or encapsulating materials. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers can be used in preparing solutions, suspensions, emulsions, syrups and elixirs. The active ingredient can be dissolved or suspended in a pharmaceutically acceptable liquid carrier such as water, an organic solvent, a mixture of both, or a pharmaceutically acceptable oil or fat. The liquid carrier can contain other suitable pharmaceutical additives such as, for example, solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

Liquid pharmaceutical compositions that are sterile solutions or suspensions can be administered by, for example, intramuscular, intraperitoneal or subcutaneous injection. Sterile solutions can also be administered intravenously. Compositions for oral administration can be in either liquid or solid form.

The crystal forms of the invention can be administered rectally or vaginally in the form of a conventional suppository. For administration by intranasal or intrabronchial inhalation or insufflation, the crystal forms of the invention can be formulated into an aqueous or partially aqueous solution, which can then be utilized in the form of an aerosol. The crystal forms of the invention can also be administered transdermally through the use of a transdermal patch containing the active compound and a carrier that is inert to the active compound, is non-toxic to the skin, and allows delivery of the agent for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the active ingredient can also be suitable. A variety of occlusive devices can be used to release the active ingredient into the blood stream such as a semipermeable membrane covering a reservoir containing the active ingredient with or without a carrier, or a matrix containing the active ingredient. Other occlusive devices are known in the literature.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

The amount of crystal form provided to a patient will vary depending upon what is being administered, the purpose of the administration, such as prophylaxis or therapy, the state of the patient, the manner of administration, and the like. In therapeutic applications, crystal forms of the invention are provided to a patient suffering from a condition in an amount sufficient to treat or at least partially treat the symptoms of the condition and its complications. An amount adequate to accomplish this is a “therapeutically effective amount” as described previously herein. The dosage to be used in the treatment of a specific case can be subjectively determined by the attending physician. The variables involved include the specific condition and the size, age, and response pattern of the patient. Generally, a starting dose is about 5 mg per day with gradual increase in the daily dose to about 150 mg per day, to provide the desired dosage level in the patient.

Crystal forms of the invention can be further processed to modulate particle size. For example, the crystal forms of the invention can be milled to reduce average crystal size and/or to prepare a sample suitable for manipulation and formulation.

In order that the invention disclosed herein may be more efficiently understood, examples are provided below. It should be understood that these examples are for illustrative purposes only and are not to be construed as limiting the invention in any manner.

### EXAMPLES

### Example 1

Preparation of Form I by Reactive Crystallization

a. 1 g of (S)-(8-(2,6-dichlorophenyl)-6-fluoro-2,3-dihydrobenzo[b][1,4]dioxin-2-yl)-methanamine was transferred into a reaction flask;
b. 7 mL (7 volumes) tBME (tert-butyl ethyl ether) at room temperature was added and with stirring to dissolve the free base;
c. About 0.1857 g of anhydrous solution of HCl in IPA was weighed (concentration: 15.8% by weight; amount: from about 1.00 to about 1.05 equivalents by mole relative to the free base);
d. 1 mL (1 volume) tBME was added to the acid and the acidic mixture was poured into the free base solution;
e. The acid vial washed with 2 mL (2 volumes) tBME and the tBME was poured into the reaction flask while stirring;
f. After nucleation, the slurry was stirred at room temperature for 5 hrs;
g. The resulting slurry was filtered and the cake washed with 1 mL (1 volume) tBME; and
h. The cake was dried over night at 50° C. and under vacuum (yield: 80-85%; Form I according to XRPD).

### Example 2

Preparation of Form Ii by Reactive Crystallization

a. 1.06 g of (S)-(8-(2,6-dichlorophenyl)-6-fluoro-2,3-dihydrobenzo[b][1,4]dioxin-2-yl)methanamine was transferred into a reaction flask;
b. 7 mL (7 volumes) of tBME (tert-butyl ethyl ether) was added at room temperature and with stirring to dissolve the free base;
c. About 0.3355 g of aqueous solution of HCl was weighed (concentration: 37.2% by weight; amount: from about 1.00 to about 1.20 equivalents by mole relative to the free base);
d. 1 mL (1 volume) tBME was added to the acid and the acidic mixture was poured into the free base solution;
e. The acid vial washed with 2 mL (2 volume) tBME and the t-BME was poured into the reaction flask while stirring;
f. After nucleation, the mixture was stirred for additional 30 minutes and then the temperature was increased to 45° C. over 30 minutes;
g. About 30-35% by volume of the solvent was evaporated;
h. The solution was cooled to 2°-25° C. over 1 hour;
i. The slurry was stirred at 2θ-25° C. for 5 hrs;
j. The slurry was filtered and the resulting cake washed with 1 mL (1 volume) tBME; and
k. The cake was dried over night at 50° C. and under vacuum (yield: 78%; Form II according to XRPD).

### Example 3

Preparation of Form Ii by Reactive Crystallization with Seeding

a. 1.06 g of (S)-(8-(2,6-dichlorophenyl)-6-fluoro-2,3-dihydrobenzo[b][1,4]dioxin-2-yl)methanamine was transferred into a reaction flask;
b. 7 mL (7 volumes) of tBME (tert-butyl ethyl ether) was added at room temperature and with stirring to dissolve the free base;
c. About 0.3355 g of aqueous solution of HCl was weighed (concentration: 37.2% by weight; amount: from about 1.00 to about 1.20 equivalents by mole relative to the free base);
d. 1 mL (1 volume) tBME was added to the acid and the acidic mixture was poured into the free base solution;
e. The acid vial washed with 2 mL (2 volume) tBME and the t-BME was poured into the reaction flask while stirring;
f. After nucleation, the mixture was stirred for additional 30 minutes and then the temperature was increased to 45° C. over 30 minutes;
g. About 30-35% by volume of the solvent was evaporated at atmospheric pressure and Form II seed was added;
h. The solution was cooled to 20-25° C. over 1 hour;
i. The slurry was stirred at 20-25° C. for 5 hrs;
j. The slurry was filtered and the resulting cake washed with 1 mL (1 volume) tBME; and
k. The cake was dried over night at 50° C. and under vacuum (yield: 78%; Form II according to XRPD).

This preparation typically resulted in higher yields than for the same preparation but without seeding (e.g., see Example 2).

### Example 4

Alternate Preparation of Form II by Reactive Crystallization with Seeding

a. 2.5108 g of (S)-(8-(2,6-dichlorophenyl)-6-fluoro-2,3-dihydrobenzo[b][1,4]dioxin-2-yl)methanamine was transferred into a reaction flask;
b. 25 mL (10 volumes) of tBME (tert-butyl ethyl ether) was added at room temperature and with stirring to dissolve the free base;
c. About 0.636 g of aqueous solution of HCl was weighed (concentration: ˜37.2% by weight; amount: from about 1.00 to about 1.20 equivalents by mole relative to the free base);
d. Crystals nucleated as Form III, then ˜3 mg Form II seed was added. A sample was taken after ˜15 minutes and the crystals were found to be Form II.
e. After ˜2 hours, the temperature was increased to ˜31-33° C. and the flask was put under vacuum to remove solvent.
f. About 50% by volume of the solvent was evaporated at reduced pressure;
g. The solution was cooled to 2°-25° C. over 1 hour;
h. 13 mL tBME was added to the slurry over 1 hour.
i. The slurry was stirred at 2θ-25° C. for ˜1 hour;
j. The slurry was filtered and the resulting cake washed with 2 mL (2 volume) tBME; and
k. The cake was dried over night at 50° C. and under vacuum (yield: 86%; Form II according to XRPD).

### Example 5

Drying of Form II to Maintain Water Content

a. 4.355 kg of wet cake (obtained, e.g., by the methods of Example 4), where the organic solvent was tBME, was placed in an oven at 25° C. and under ˜25-30 mmHg vacuum. At the same time a tray of distilled water was placed in the oven to keep the oven atmosphere humid.
b. The cake was taken out after 4 hours resulting in 3.199 kg of Form II with less than 0.02% tBME.

### Example 6

Preparation of Form II from Form I

a. 1 g of Form I was transferred into a reaction flask;
b. 4 mL (4 volumes) tBME were added with stirring;
c. 0.21 g water were added (about 4 equivalents by mole relative to Form I);
d. The temperature was increased to 45-50° C. while stirring to dissolve the crystals;
e. 6 mL (6 volumes) were added tBME at 45-50° C.;
f. The solution was cooled to 25° C. over 1.5 hr;
g. About 30-40% by volume of the solvent was evaporated and monohydrate seed was added;
h. The mixture was stirred for 5 hrs and then filtered;
i. The filter cake washed with 1 mL (1 volume); and
j. The cake was dried over night at 50° C. and under vacuum (yield: 75-82%; Form II according to XRPD).

### Example 7

Preparation of Form Iii

37 mg of Form I of (S)-(8-(2,6-dichlorophenyl)-6-fluoro-2,3-dihydrobenzo[b][1,4]dioxin-2-yl)methanamine hydrochloride salt was combined with 2 mL tBME:water (100:0.5 v/v). The temperature was increased to 50° C., cooled to room temperature naturally, stirred overnight at room temperature, and then filtered. The resulting solid was characterized as Form III according to XRPD.

### Example 8

Preparation of Form I from a Mixture of Form I and Form Iii in an Organic Solvent

A mixture of Form I and Form III (2θ-50 wt % of Form III) was slurried in a solvent (see Table 5) with stirring at room temperature for about 2 days. The slurry was then filtered at room temperature. The solid was dried at 50° C. under vacuum for overnight. The solid obtained by the filtration was characterized by XRPD both before and after the drying at 50° C. The results are summarized in Table 5 below.

TABLE 5
Crystal form of the solid obtained from the slurry

| | Initial | Final Form, | Final Form, |
|---|---|---|---|
| Solvent | Form | Slurry @ RT | Slurry @ 50° C. |
| Ethanol | I & III | I | Not performed |
| isopropyl alcohol (IPA) | I & III | I | I |
| Ethyl acetate | I & III | I | I |
| tBME | I & III | I | I |
| Acetonitrile | I & III | I | I |
| Toluene | I & III | Small endo. peak at 68.5° C. by | I |
| | | DSC (I and minor III) | |
| Isopropyl acetate | I & III | Tiny endo. peak at 48.5° C. by | Tiny endo. peak at 59.9° C. |
| | | DSC (I and minor III) | by DSC (I and minor III) |
| 3.74 wt % water in IPA | I & III | I | Not performed |

### Example 9

Preparation of Form I from Mixture of Form I and Form Iii by Heating

Method A

A mixture of Form I and Form III (2θ-50 wt % of Form III) was heated to 110° C. at ambient pressure and kept at this temperature and pressure for 4 hours. The resulting solid was characterized as anhydrous Form I according to XRPD.

Method B

A mixture of Form I and Form III (2θ-50 wt % of Form III) was placed in an oven at 50° C. and under vacuum for 72 hours. The resulting solid was characterized as anhydrous Form I according to XRPD.

### Example 10

Crystal form Stability Assessment of Forms I, II and III

A. Solution mediated transformation

As shown in Table 6, a series of experiments were carried out in which different forms (Forms I, II and III) or their mixtures were slurried in water or an organic solvent with stirring (with magnetic stirrer) for a period of time. The mixtures were then filtered and the solids obtained were characterized by one or more methods of XRPD, DSC and TGA. Table 6 shows that Form II (monohydrate) is stable in water, and that Form I (anhydrous) is stable in organic solvents. Form III (hemihydrate) is less stable under some conditions.

TABLE 6
Stability Experiments at 25° C.

| | | | | | Final form |
|---|---|---|---|---|---|
| | | | | Sample from | Dried @ 50° C. |
| Exp | Original | Reslurry | Stirring | wet filter | under |
| # | form | solvent | time | cake | vacuum, overnight |
| 1 | I | Water | 5 mins | II | — |
| 2 | I | Water | 30 mins | II | II |
| 3 | II | tBME | 30 mins | I + II | — |
| 4 | II | Ethyl | 30 mins | I | I |
| | | acetate | | | |
| 5 | I + III | Ethyl | 30 mins | I | I |
| | | acetate | | | |

B. Thermal Stability Assessment

Forms I, II and III were placed in oven at 50° C. and under vacuum to assess their thermal stability. Results are provided in Table 7. Thermally, Forms I and II are more stable than Form III under the tested conditions (in terms of retention of crystal lattice integrity).

TABLE 7
Thermal stability of crystal forms under vacuum and 50° C.

| Ex # | Original Form | Duration, hr | Final Form |
|---|---|---|---|
| 1 | Form I | 48 | Form I |
| 2 | Form II | 72 | Form II |
| 3 | Form III | 20 | Form I + Form III |

C. Grinding Test

Samples of Form I and Form II were manually ground and the resulting powders were characterized by one or more methods of XRPD, DSC and TGA. The results are summarized in Table 8. This test showed that some of Form I is changed into Form III during the grinding under ambient conditions indicating that Form I absorbs water during the grinding. However, Form II did not lose its crystal structure (the integrity of crystal lattice) under the same situation.

TABLE 8
Grinding test on Forms I and II

| Exp # | Original solid form | After grinding |
|---|---|---|
| 1 | Form I | Form I + Form III |
| 2 | Form II | Form II |

Various modifications of the invention, in addition to those described herein, will be apparent to those skilled in the art from the foregoing description. Such modifications are also intended to fall within the scope of the appended claims. Each reference, including all patents, patent applications, and journal literature, cited in the present application is incorporated herein by reference in its entirety.

What is claimed is:

1. A crystal form (Form I) of (S)-(8-(2,6-dichlorophenyl)-6-fluoro-2,3-dihydrobenzo[b][1,4]dioxin-2-yl)methanamine hydrochloride having a powder X-ray diffraction pattern comprising a characteristic peak, in terms of 2θ, at about 10.3° and at least one characteristic peak, in terms of 2θ, selected from about 23.1° and about 25.7°.

2. The crystal form of claim 1 wherein said powder X-ray diffraction pattern comprises characteristic peaks, in terms of 2θ, at about 10.3°, about 23.1°, and about 25.7°.

3. The crystal form of claim 1 having a powder X-ray diffraction pattern substantially as shown in FIG. 1.

4. The crystal form of claim 1 having a differential scanning calorimetry endotherm at about 207° C.

5. The crystal form of claim 1 having a scanning calorimetry thermogram substantially as shown in FIG. 2.

6. A composition comprising the crystal form of claim 1 and a pharmaceutically acceptable carrier.

7. A process for preparing the crystal form of claim 1 comprising precipitating said crystal form from a solution comprising (S)-(8-(2,6-dichlorophenyl)-6-fluoro-2,3-dihydrobenzo[b][1,4]dioxin-2-yl)methanamine hydrochloride salt and a crystallizing solvent, wherein said solution comprises about 1% or less of water by weight.

8. A method for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease comprising administering to a patient a therapeutically effective amount of a crystal form of claim 1.

9. A method for treating bipolar disorders, depressive disorders, mood episodes, anxiety disorders, adjustment disorders, or eating disorders comprising administering to a patient a therapeutically effective amount of a crystal form of claim 1.

10. A method for treating pain, urinary incontinence, substance abuse, addiction to alcohol and other drugs, epilepsy, sleep disorders, migraines, sexual dysfunction, gastrointestinal disorders, or obesity comprising administering to a patient a therapeutically effective amount of the crystal form of claim 1.

11. A hydrate of (S)-(8-(2,6-dichlorophenyl)-6-fluoro-2,3-dihydrobenzo[b][1,4]dioxin-2-yl)methanamine hydrochloride.

12. The hydrate of claim 11 where said hydrate is crystalline.

13. A crystal form (Form II) of a hydrate of (S)-(8-(2,6-dichlorophenyl)-6-fluoro-2,3-dihydrobenzo[b][1,4]dioxin-2-yl)methanamine hydrochloride having a powder X-ray diffraction pattern comprising a characteristic peak, in terms of 2θ, at about 9.5° and at least one characteristic peak, in terms of 2θ, selected from about 15.4°, about 26.1°, and about 26.3°.

14. The crystal form of claim 13 wherein said powder X-ray diffraction pattern comprises characteristic peaks, in terms of 2θ, at about 9.50, about 15.4°, and about 26.1°.

15. The crystal form of claim 13 having a powder X-ray diffraction pattern substantially as shown in FIG. 6.

16. The crystal form of claim 13 having a scanning calorimetry thermogram comprising an endotherm at about 94° C., an endotherm at about 112° C. and an endotherm at about 207° C.

17. The crystal form of claim 13 having a scanning calorimetry thermogram substantially as shown in FIG. 7.

18. The crystal form of claim 13 wherein the molar ratio of the hydrate water to (S)-(8-(2,6-dichlorophenyl)-6-fluoro-2,3-dihydrobenzo[b][1,4]dioxin-2-yl)methanamine hydrochloride is from about 0.3 to about 1.0.

19. A composition comprising the crystal form of claim 13 and a pharmaceutically acceptable carrier.

20. A process for preparing the crystal form of claim 13 comprising precipitating said crystal form from a solution comprising (S)-(8-(2,6-dichlorophenyl)-6-fluoro-2,3-dihydrobenzo[b][1,4]dioxin-2-yl)methanamine hydrochloride and a crystallizing solvent, wherein said solution comprises at least about 2.5% water by weight.

21. A method for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease comprising administering to a patient a therapeutically effective amount of a crystal form of claim 13.

22. A method for treating bipolar disorders, depressive disorders, mood episodes, anxiety disorders, adjustment disorders, or eating disorders comprising administering to a patient a therapeutically effective amount of a crystal form of claim 13.

23. A method for treating pain, urinary incontinence, substance abuse, addiction to alcohol and other drugs, epilepsy, sleep disorders, migraines, sexual dysfunction, gastrointestinal disorders, or obesity comprising administering to a patient a therapeutically effective amount of the crystal form of claim 13.

24. A crystal form (Form III) of a hydrate of (S)-(8-(2,6-dichlorophenyl)-6-fluoro-2,3-dihydrobenzo[b][1,4]dioxin-2-yl)methanamine hydrochloride having a powder X-ray diffraction pattern comprising a characteristic peak, in terms of 2θ, at about 9.5° and at least one characteristic peak, in terms of 2θ, selected from about 14.3°, and about 23.9°.

25. The crystal form of claim 24 wherein said powder X-ray diffraction pattern comprises characteristic peaks, in terms of 2θ, at about 9.5°, about 14.3°, and about 23.9°.

26. The crystal form of claim 24 having a powder X-ray diffraction pattern substantially as shown in FIG. 11.

27. The crystal form of claim 24 having a scanning calorimetry thermogram comprising an endotherm at about 112° C. and an endotherm at about 207° C.

28. The crystal form of claim 24 having a scanning calorimetry thermogram substantially as shown in FIG. 12.

29. The crystal form of claim 24 wherein the molar ratio of the hydrate water to (S)-(8-(2,6-dichlorophenyl)-6-fluoro-2,3-dihydrobenzo[b][1,4]dioxin-2-yl)methanamine hydrochloride is from about 0.3 to about 0.5.

30. A composition comprising the crystal form of claim 24 and a pharmaceutically acceptable carrier.

31. A process for preparing the crystal form of claim 24 comprising precipitating said crystal form from a solution comprising (S)-(8-(2,6-dichlorophenyl)-6-fluoro-2,3-dihydrobenzo[b][1,4]dioxin-2-yl)methanamine hydrochloride and water.

32. A method for treating schizophrenia, schizophreniform disorder, schizoaffective disorder, delusional disorder, substance-induced psychotic disorder, L-DOPA-induced psychosis, psychosis associated with Alzheimer's dementia, psychosis associated with Parkinson's disease, psychosis associated with Lewy body disease, dementia, memory deficit, or intellectual deficit disorder associated with Alzheimer's disease comprising administering to a patient a therapeutically effective amount of a crystal form of claim 24.

33. A method for treating bipolar disorders, depressive disorders, mood episodes, anxiety disorders, adjustment disorders, or eating disorders comprising administering to a patient a therapeutically effective amount of a crystal form of claim 24.

34. A method for treating pain, urinary incontinence, substance abuse, addiction to alcohol and other drugs, epilepsy, sleep disorders, migraines, sexual dysfunction, gastrointestinal disorders, or obesity comprising administering to a patient a therapeutically effective amount of the crystal form of claim 24.
